(19) United States
(12) Patent Application Publication
(10) Pub. No.: US 2011/0082102 A1
(43) Pub. Date: Apr. 7, 2011

(54) USE OF PREBIOTIC GALACTO-OLIGOSACCHARIDES IN THE TREATMENT OF INTESTINAL INFLAMMATION

(76) Inventors: Georgios Tzortzis, Reading Berkshire (GB); Jelena Vulevic, Reading Berkshire (GB); Francesco Attanasio, Reading Berkshire (GB)

(21) Appl. No.: 12/995,134

(22) PCT Filed: May 26, 2009
(86) PCT No.: PCT/GB09/01302

§ 371 Date: Dec. 10, 2010

(30) Foreign Application Priority Data
May 30, 2008 (GB) 0809921.0

(51) Int. Cl. A61K 31/715; A61P 29/00

(52) U.S. Cl. 514/53

(57) ABSTRACT

The present invention relates to the use of an oligosaccharide, in particular a non-digestible oligosaccharide, composition in the prevention or treatment of inflammation, in particular intestinal inflammation.

The present invention relates to the use of an oligosaccharide, in particular a non-digestible oligosaccharide composition in the prevention or treatment of inflammation, in particular in the prevention or treatment of intestinal inflammation. The composition comprises a mixture of galactooligosaccharides. Galactooligosaccharides are non-digestible carbohydrates, which are resistant to mammalian gastrointestinal digestive enzymes but are fermented by specific colonic bacteria.

The human gut flora comprises pathogenic, benign and beneficial microbial genera. A predominance of the former can lead to intestinal disorders that can be both acute (eg gastroenteritis) and chronic (eg inflammatory bowel disease and some intestinal cancers). Attempts have been made to influence the balance of the gut flora in favour of beneficial microorganisms, such as the bifidobacteria, by adding one or more such microbial strains to an appropriate food vehicle. Such a live microbial feed supplement is known as a probiotic. However, it is difficult to guarantee the survival of live bacteria in foods and also after digestion.

An alternative approach to dietary manipulation of the gut microflora is the use of a prebiotic, which is defined as a non-digestible food ingredient that beneficially affects the host by selectively stimulating the growth and/or activity of one or a limited number of bacteria in the colon, thereby resulting in an improvement in the health of the host.

Prebiotics have been shown to have an indirect protective effect in a number of inflammatory conditions such as inflammatory bowel disease (IBD). It has been found in some IBD patients that the adaptive immune system is hyper-responsive to commensal intestinal flora (see Guarner F, Malagelada J R, Best Pract. Res. Clin. Gatroenterol., (2003); 17; 793-804). As a result, prebiotics have been used to enhance beneficial gut microflora which has helped to prevent a relapse in the disease (see Sartor R D., Gastroenterology, (2004), 126, 1620-1633).

One group of compounds that is classified as prebiotics are the galactooligosaccharides. These are galactose-containing oligosaccharides of the form Glc β 1-4 [Gal β 1-6]ₙ where n=2-5 and are produced from lactose syrup using the transgalactosylase activity of the enzyme β-galactosidase (Crittenden, (1999) Probiotics : A Critical Review, Tannock, G. (ed) Horizon Scientific Press, Wymondham, pp 141-156).

EP 1 644 482 discloses a novel strain of Bifidobacterium bidifum that produces a galactosidase enzyme activity that converts lactose to a novel mixture of galactooligosaccharides. This mixture of galactooligosaccharides has been shown to have prebiotic properties and to increase the population of the beneficial bacteria bifidobacteria and lactobacilli.

It has now been found unexpectedly that a composition comprising the mixture of galactooligosaccharides disclosed in EP 1 644 482 can directly modulate the inflammatory response of the mammalian intestinal mucosa. In particular, it attenuates the pro-inflammatory chemokine response in the presence of inflammatory agents.

According to the invention there is provided a prebiotic composition for use in the prevention or treatment of inflammation, preferably in the prevention or treatment of intestinal inflammatory disorders.

The prebiotic composition is a galactooligosaccharide mixture. This mixture comprises a disaccharide Gal-Gal, a trisaccharide Gal-Gal-Glc, a tetrasaccharide Gal-Gal-Gal-Glc and a pentasaccharide Gal-Gal-Gla-Gal-Glc, where Gal represents a galactose residue and Glc represents a glucose residue.

Preferably, the galactooligosaccharide mixture comprises disaccharides Gal (β 1-3) Glc; Gal (β 1-3)-Gal; Gal (β 1-6)-Gal; Gal (α 1-6)-Gal; trisaccharides Gal (β 1-6)-Gal (β 1-4)-Glc; Gal (β 1-3)-Gal (β 1-4)-Glc; tetrasaccharide Gal (β 1-6)-Gal (β 1-6)-Gal (β 1-4)-Glc and pentasaccharide Gal (β 1-6)-Gal (β 1-6)-Gal (β 1-6)-Gal (β 1-4)-Glc. This mixture of galactooligosaccharides is marketed commercially under the name Bimuno (Registered Trade mark) and is available from Clasado Ltd (Milton Keynes, UK).

Enterocytes form a single polarized epithelial layer separating the luminal environment from the host. They are active contributors to the host defence. Their innate immune response to any inflammatory stimuli is primarily responsible for rapidly regenerating the barrier function of the epithelium. The epithelium can be induced to express pro-inflammatory cytokines and chemokines that begin the process of recruiting innate immune cells such as neutrophils to the damaged mucosa, if necessary. For example, pro-inflammatory chemokines, such as IL-8, can be stimulated during an immune response by epithelial cells and by macrophages to recruit neutrophils and PMN's (polymorphonuclear leukocytes) to the inflamed mucosa. Macrophage Inflammatory Protein-3α (MIP-3α) or CCL20 is another chemokine that elicits the adaptive immune system by activating the lymphocytes and dendritic cells through activation of chemokine receptor CCR6. The IL-8 and MIP-3α (CCL20) induction indicates the degree of response to an inflammation challenge.

The effect of the galactooligosaccharide mixture, known as Bimuno, on the inflammatory response of different adult colonic cell culture models has been studied. It was found unexpectedly that Bimuno at physiologic concentrations, attenuates the pro-inflammatory chemokine response induced by TNF-α inflammatory stimulus in intestinal epithelial cells, ie human enterocytes.

It was also found that Bimuno reduced the translocation of NF-κB p65 protein and thus could be useful in the treatment of such diseases as asthma, neurodegeneration, ischemia/reperfusion injury, hepatitis, glomerulonephritis, rheumatoid arthritis, allergies, type II diabetes, obesity, sepsis, autoimmunity, multiple sclerosis and atherosclerosis.

The galactooligosaccharide composition known as Bimuno is a freeze-dried powder of the mixture of galactooligosaccharides. Bimuno comprises 49% w/w of galactooligosaccharide. The remainder of the composition may comprise non-active components such as glucose, lactose, acacia gum and citric acid. It may be administered to a patient suffering from an inflammatory disorder, for example an intestinal inflammatory disorder, daily in an effective dose of from 1.35 g to 9.6 g of galactooligosaccharide in 2.75 to 20 g of the powder, preferably from 1.96 to 4.9 g of galactooligosaccharide in 4 to 10 g of powder, most preferably 2.7 galactooligosaccharide in 5.5 g of the powder. This can be taken in one single dose or two separate doses several hours apart. The Bimuno product may be added to a hot drink or sprinkled on food.

In order to prevent inflammation the Bimuno powder may be administered to an individual in an effective daily dose of 2 to 15 g, preferably 2.5 to 10 g, most preferably 5.5 g.

According to another aspect of the invention, there is provided a method for the treatment and/or prevention of inflammation, such as intestinal inflammation comprising orally administering to a mammal an effective amount of an oligosaccharide composition.

The present invention will be further described by way of reference to the following examples and figures.

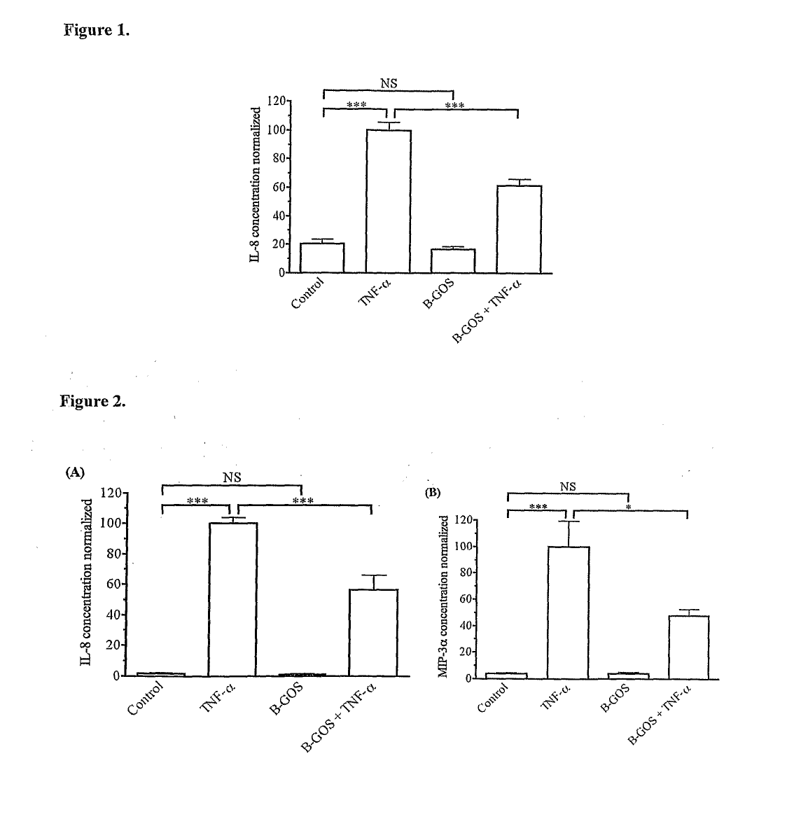

FIG. 1 shows the effect of B-GOS on the TNF-α induced IL-8 secretion in T84 cells;

FIGS. 2(A) and (B) show the effect of B-GOS on TNF-α induced IL-8 and MIP-3α secretion in NCM-460 cells;

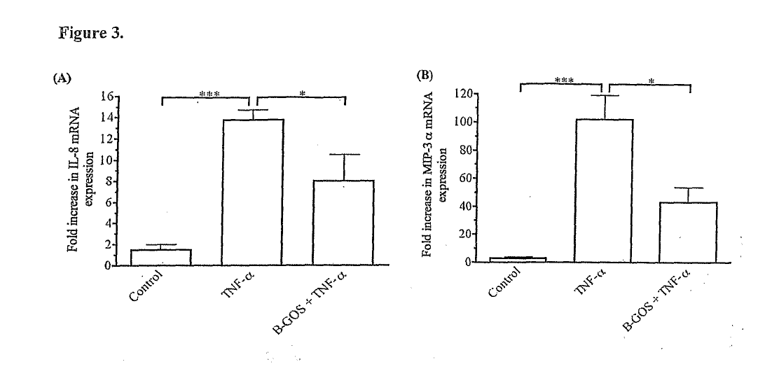

FIGS. 3(A) and (B) show the effect of B-GOS on the expression of IL-8 and MIP-3α mRNA in TNF-α treated NCM-460 cells;

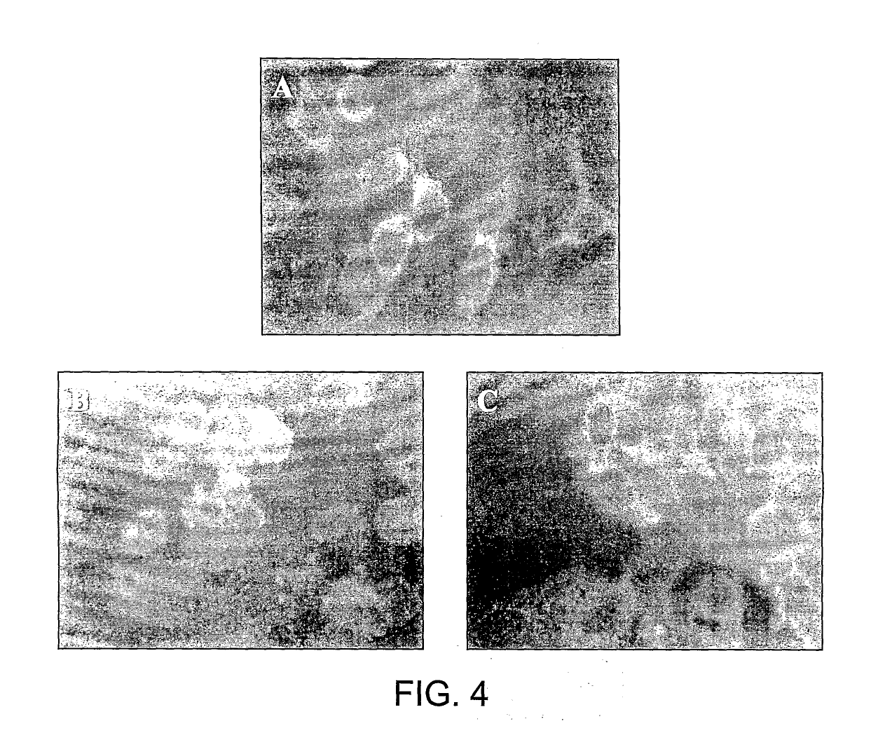

FIGS. 4(A), (B) and (C) show the effect of B-GOS on the translocation of NF-κB p65 protein into the nuclei of TNF-α treated NCM-460 cells;

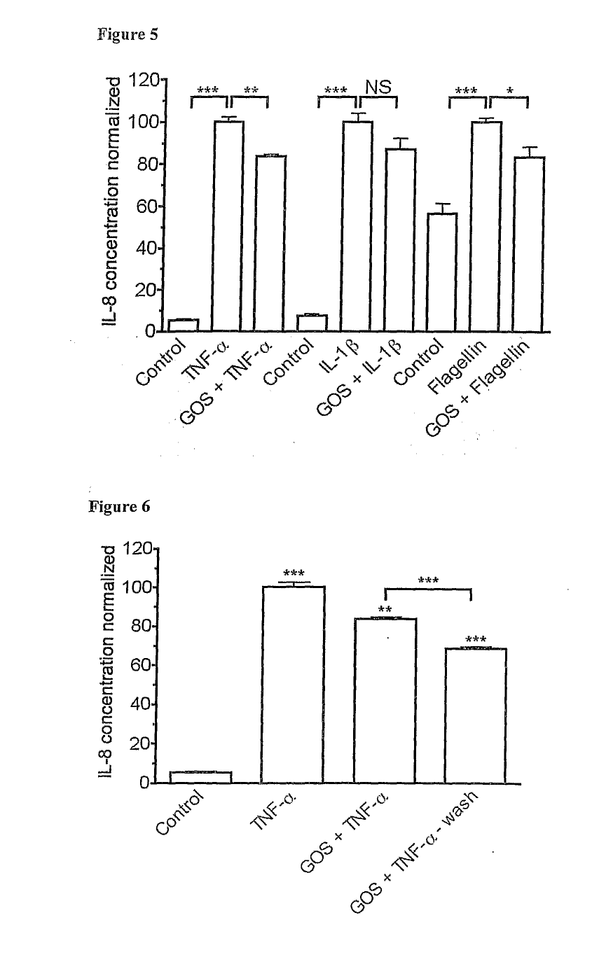

FIGS. 5 and 6 show the effect of B-GOS on TNF-α induced IL-8 secretion in NCM-460 cells; and

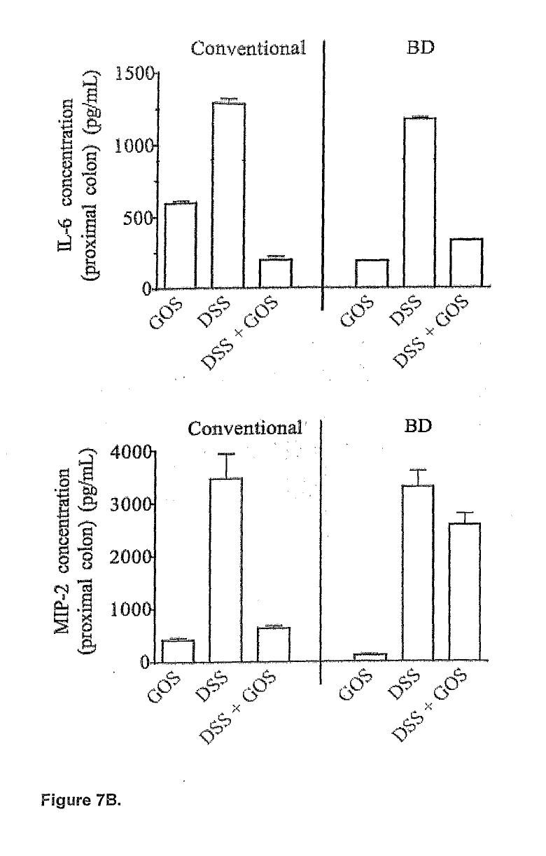

FIGS. 7(A) and (B) show the effect of B-GOS on IL-6 and MIP-2 secretion in DSS treated mice.

### Example 1

### Effect of Galactooligosaccharides on Cytokine Secretion

Intestinal epithelial cells were grown to confluence in 24-well plates from an initial concentration of 5×10⁵ cells/mL. When the cells reached 70% confluence, they were treated in quadruplicate as follows: (i) negative control, (ii) TNF-α (10 ng/mL) positive control, (iii) B-GOS (5 g/L) and (iv) TNF-α (10 ng/mL) with B-GOS (5 g/L). A concentration of 5 g/L of oligosaccharides was used since this is the physiological concentration of oligosaccharides found in human milk. After 16 hours, supernatants were collected and stored at −20° C. for IL-8 and MIP-3α secretion to be determined later by ELISA. In following experiments, TNF-α was replaced by IL1β or flagellin.

Quantitation of IL-8. The IL-8 concentration was measured by ELISA as described previously (Claud E C, Savidge T, Walker W A 2003 Modulation of human intestinal epithelial cell IL-8 secretion by human milk factors. Pediatr Res 53:419-425). Briefly, each well of a 96-well high bond plate (Nunc Immulon, Fisher Scientific, Middletown, Va., USA) was coated overnight with 100 μL of 3 μg/mL mouse anti-human IL-8 monoclonal antibody, washed three times with 200 μL of 1% BSA in PBS and incubated with 100 μL of samples at 37° C. for one hour. The wells were then washed three times and incubated with 100 μL of 0.1 μg/mL biotin-labelled mouse antihuman IL-8 antibody for one hour. After another wash, each well was incubated with 100 μL horseradish peroxidase, washed again before incubating with 100 μL O-phenylenediamine dihydrochloride and hydrogen peroxide. The reaction was stopped with 100 μL 2N H2SO4 and the absorbance was read at 490 nm. The concentration of IL-8 in the samples was calculated from the IL-8 standard curve.

Quantitation of MIP-3α. The amount of MIP-3α secretion was measured by ELISA similar to IL-8, except that the plate was coated overnight with 100 μL 2.0 μg/mL mouse anti-human MIP-3α monoclonal antibody. The detection antibody, biotin-labelled mouse antihuman MIP-3α antibody, was used as detection antibody at a concentration of 50 ng/mL with a volume of 100 μL. The concentration of MIP-3α in the samples was calculated from the MIP-3α standard curve.

Cell viability assay. B-GOS cytotoxicity was investigated using the trypan blue exclusion test. NCM-460 cells were grown on coverslips from an initial concentration of 2×105 cells/mL. The cells were treated in triplicate with: B-GOS (5 g/L) or control medium. After 16 hours, NCM-460 cells were assayed for cell viability by trypan blue exclusion assay (Raimondi F, Crivaro V, Capasso L, Maiuri L, Santoro P, Tucci M, Barone M V, Pappacoda S, Paludetto R 2006 Unconjugated bilirubin modulates the intestinal epithelial barrier function in a human-derived in vitro model. Pediatr Res 60:30-33). There was no significant effect of B-GOS on the viability of the cells at this concentration.

Effect of B-GOS on induction of cytokine transcription. NCM-460 cells were grown to confluence in 6-well plates from an initial concentration of 5×105 cells/mL. When the cells reached 70% confluence, they were treated as follows in quadruplicate: (i) negative control, (ii) TNF-α or IL1β or flagellin (10 ng/mL) positive control, and (iii) TNF-α or IL1β or flagellin (10 ng/mL) with B-GOS (5 g/L). After 18 hours, total cellular RNA was isolated by Trizol-chloroform extraction. Using the SuperScript III Platinum SYBR Green One-Step qRT-PCR kit, the mRNA expression of IL-8, MIP-3α and MCP-1 was measured on a MJ Opticon 2 and standardized to mRNA expression of GAPDH.

Effect of B-GOS on NF-κB translocation. NCM-460 cells were grown to 70% confluency on cover slips and treated in duplicate for 10 or 30 minutes as follows: (i) negative control, (ii) TNF-α (10 ng/mL) positive control, and (iii) TNF-α (10 ng/mL) with B-GOS (5 g/L). The medium was removed and the cells were fixed in 4% paraformaldehyde. After permeabilization with methanol and blocking with 10% goat serum in 0.25% BSA in TBS, the cells were probed with rabbit anti-human NF-κB p65 polyclonal antibody. After washing, the cells were incubated with CyTM 3-conjugated goat anti-rabbit antibody. The cover slips were then washed and mounted on a glass slide to be visualized under the microscope (Nikon Eclipse TE2000-S).

### Materials

TNF-α cytokine, IL1β, flagellin, streptavidin-HRP and human CCL20-MIP-3α ELISA development kits (Quantikine) were obtained from R&D Systems (Minneapolis, Minn., USA). Antihuman IL-8 and mouse anti-human IL-8 antibodies were obtained from Pierce Endogen (Woburn, Mass., USA). O-phenylenediamine tablets were obtained from Pierce (Rockford, Ill., USA). Trizol, SuperScript III Platinum SYBR Green One-Step qRT-PCR kits and other reagents necessary for qRT-PCR were obtained from Invitrogen (Carlsbad, Calif., USA). DMEM/F12 medium, CMRL medium, penicillin, streptomycin and Hepes buffer were obtained from Gibco-Invitrogen (Carlsbad, Calif., USA). Fetal bovine serum was obtained from Atlanta Biologicals (Lawrenceville, Ga., USA). M3D was obtained from Incell Corp. (San Antonio, Tex., USA). Rabbit anti-human NF-κB (p65) polyclonal antibody was obtained from Calbiochem (Gibbstown, N.J., USA). CyTM 3-conjugated F(ab')2 fragment goat anti-rabbit IgG was obtained from Jackson ImmunoResearch (West Grove, Pa., USA). All other reagents for immunofluorescence were obtained from Vector Lab (Burlingame, Calif., USA). All other reagents were of analytical or molecular biological grade from Sigma-Aldrich (St. Louis, Mo., USA).

B-Galacto-oligosaccharides B-GOS. Bimuno® was supplied by Clasado Ltd., Milton Keynes, UK.

Intestinal Epithelial Cell lines. Two adult human intestinal epithelial culture models were used in these studies: T84 and NCM-460 cells are transformed and untransformed colonic epithelial cells, respectively. Cells were cultured in Falcon cell culture dishes at 37° C. with 95% O2 and 5% CO2 atmosphere saturated with water vapour. T84 culture medium consisted of DMEM/F12 supplemented with FBS (5%), Hepes buffer, glutamine, non-essential amino acids, penicillin and streptomycin (12). NCM-460 culture medium consisted of M3D medium supplemented with FBS (10%), penicillin and streptomycin as described previously (13).

Statistical analysis. Induction of cytokines was standardized to the positive control with error bars representing standard error (SE). Comparisons between groups were performed using a two-tailed Student's t test. Gene expression data obtained by qRT-PCR was expressed as the mean with SE. Comparisons between groups were performed using a Student's two-tailed t test after logarithmic transformation. A p value <0.05 was considered statistically significant and indicated by an asterisk (*), a p value <0.01 was indicated by two asterisks (**) and a p value <0.001 was indicated by three asterisks (***).

### Results

### Effect of Galacto-Oligosaccharides B-GOS on Cytokine Secretion in T84 Cells (FIG. 1)

TNF-α-induced IL-8 secretion in T84 cells was normalized to 100% to allow for comparison between 4 independent experiments. The untreated T84 cells had a basal IL-8 secretion at 20.5%. Upon TNF-α stimulation, IL-8 secretion was significantly increased by 4.9 fold (p<0.001).

To determine the effect of B-GOS, T84 cells were stimulated with or without TNF-α in the presence of galacto-oligosaccharides B-GOS (5 g/L). B-GOS-treated T84 cells secreted IL-8 at 16.4%. This was not significantly different from basal level of untreated T84 cells. Upon stimulation with TNF-α, B-GOS significantly attenuated IL-8 secretion by 38.5% (p<0.001).

### Effect of Galacto-Oligosaccharides B-GOS on Cytokine Secretion in NCM-460 Cells (FIG. 2, FIG. 5, FIG. 6)

TNF-α-induced IL-8 and MIP-3α secretion in NCM-460 cells was normalized to 100% to allow for comparison between 4 independent experiments. The untreated NCM-460 cells had a basal IL-8 and MIP-3α secretion at 1.7% and 4.0% respectively. Upon TNF-α stimulation, IL-8 and MIP-3α secretion was significantly increased by 58.8 fold (p<0.001) (FIG. 2A) and 25.0 fold (p<0.001) (FIG. 2B) respectively.

To determine the effect of B-GOS, NCM-460 cells were stimulated with or without TNF-α in the presence of galacto-oligosaccharides B-GOS (5 g/L). B-GOS-treated NCM-460 cells secreted IL-8 and MIP-3α at 1.1% and 3.9% respectively; this was not significantly different from basal level of untreated NCM-460 cells. Upon stimulation with TNF-α, B-GOS significantly attenuated IL-8 and MIP-3α secretion by 43.5% (p<0.001) (FIG. 2A) and 52.1% (p<0.05) (FIG. 2B) respectively. In the same manner, when NCM-460 cells were prewashed with B-GOS prior to TNF-α stimulation, the secretion of IL8 was significantly reduced by 32% (p<0.001) even in the absence of B-GOS (FIG. 6). This suggests that constituents of the B-GOS mixture interact with epithelial receptors such as toll-like receptors (TLR) to prevent inflammatory stimulation of the cell.

Similarly upon stimulation with flagellin, B-GOS significantly attenuated IL-8 secretion by 21.5% (p<0.05) (FIG. 5). No effect could be observed upon stimulation with

To determine if B-GOS is cytotoxic, NCM-460 cells were incubated for 16 hours with or without B-GOS. B-GOS did not affect cell viability as determined by a trypan blue exclusion assay as described in the methods.

### Effect of Galacto-Oligosaccharides B-GOS on Cytokine Expression (FIG. 3)

Total RNA of TNF-α treated NCM-460 cells was isolated and assayed for IL-8, MIP-3α and MCP-1 mRNA expression by qRT-PCR. Upon stimulation with TNF-α, IL-8 and MIP-3α mRNA expression was significantly increased by 12.2 fold (p<0.001) (FIG. 3A) and 99.4 fold (p<0.001) (FIG. 3B) respectively. No change in MCP-1 mRNA expression was observed between any of the treatment (p=0.19) (data not shown). To determine the effect of B-GOS, NCM-460 cells were stimulated with TNF-α in the presence of B-GOS (5 g/L). Galacto-oligosaccharides B-GOS significantly attenuated TNF-α-induced IL-8 and MIP-3α mRNA expression by 5.7 fold (p<0.05) (FIG. 3A) and 58.9 fold (p<0.05) (FIG. 3B) respectively. The MCP-1 mRNA expression was reduced by B-GOS but did not reach significance (p=0.06) (data not shown).

### Effect of Galacto-Oligosaccharides B-GOS on NF-κB Translocation (FIG. 4)

Adult colonic NCM-460 cells were treated with TNF-α (10 ng/mL) and assayed for nuclear translocation of NF-κB p65 protein. In the vehicle treated control cells (FIG. 4A), staining for NF-κB p65 was predominantly in the cytoplasm and the nucleus was free of p65 protein. NF-κB p65 is clearly translocated into the nuclei after 30 minutes upon stimulation with TNF-α (FIG. 4B).

However in the presence of B-GOS, TNF-α-induced NF-KB translocation was partially inhibited at 30 minutes (FIG. 4C).

### EXAMPLE 2

### In Vivo Study of the Effect of B-GOS in Dextran Sulphate Sodium Induced Colitis Mouse Model

Material Methods

Two groups (n=24 each) of adult C57BL/6 mice (Jackson Laboratories, Bar. Harbour, Me., USA), conventionally raised (CR) and bacteria-depleted (BD) mice, were used to induce colitis. All animals were housed within a 12-h light/dark cycle and had access to mouse chow and water ad libitum.

At 6 weeks of age, conventionally colonised mice were housed under conventional conditions with untreated water (CR group) whilst mice in the BD group were fed an antibiotic cocktail in their drinking water for 2 weeks. Kanamycin (8 mg/ml), Gentamicin (0.7 mg/ml), Colistin (34,000 U/ml), Metronidazole (4.3 mg/ml) and Vanacomycin (0.9 mg/ml) comprised the antibiotic cocktail. Concentrations of antibiotics in the water were calculated based on the average water consumed by age group.

At 8 weeks of age, intestinal colitis was induced by feeding 3.5% DSS (Dextran Sulphate Sodium) (MP Biomedicals, Aurora, Ohio, USA) in drinking water for 5 days in all mice of both groups (CR and BD). At 10 weeks of age, half the mice of each group started receiving Bimuno (5 g/L) for 7 days. At the end of week 10, the animals were euthanized and their colons were harvested for analysis.

### Cytokine Measurements

Murine IL-6 and MIP-2 cytokines were analysed by ELISA (Quantikine, R&D Systems, Minn., USA) on colon tissue homogenates according to manufacturer's instructions. Briefly, the proximal colons for each group were collected and homogenised with PBS homogenising buffer containing 1% Triton X-100 supplemented with a cocktail of protease inhibitors. The homogenised solutions were centrifuged at 12,000 rpm for 10 min, and the supernatants were separated into aliquots and stored at −70° C.

### Results

The capacity of Bimuno to reduce the injury and inflammation of DSS colitis in both groups of mice (CR and BD) compared to the control mice (no Bimuno administration) was determined.

In conventional DSS-treated mice, IL-6 and MIP-2 secretion was significantly induced by 2.2 (p<0.0001) and 8.3 fold (p<0.0001) respectively. Bimuno significantly attenuated IL-6 and MIP-2 secretion by 6.6 (p<0.0001) and 5.5 fold (p<0.0001).

In BD DSS-treated mice, IL-6 and IP-2 secretion was significantly induced by 6.2 (p<0.0001) and 27.2 fold (p=0.0005) respectively. Bimuno significantly attenuated IL-6 secretion by 3.6 fold (p<0.0001). MIP-2 secretion was reduced by 1.3 fold but this was found to be not significant (p=0.126).

In summary, conventional DSS-treated mice developed colitis compared to the untreated group. DSS-treated conventional mice supplemented with Bimuno had significantly reduced markers of inflammation (IL-6 and MIP-2) and alleviated symptoms of colitis. The same effect was observed in bacteria-depleted DSS-treated mice. This implies that the observed reduction in inflammation due to Bimuno is not mediated through the microflora. Bimuno has a direct immune-modulatory effect on the intestinal epithelium in DSS colitis.

1. An oligosaccharide composition for use in the prevention or treatment of inflammation.

2. The oligosaccharide composition according to claim 1, which comprises a mixture of prebiotic oligosaccharides.

3. The composition according to claim 2, which comprises a mixture of galactooligosaccharides.

4. The composition according to claim 3, wherein the mixture of galactooligosaccharides comprises a disaccharide Gal-Gal, a trisaccharides Gal-Gal-Glc, a tetrasaccharide Gal-Gal-Gal-Glc and a pentasaccharide Gal-Gal-Gal-Gal-Glc.

5. The composition according to claim 4, wherein the galactooligosaccharide mixture comprises disaccharides Gal (β 1-3)-Glc, Gal (β 1-3)-Gal, Gal (β 1-6)-Gal, Gal (α 1-6)-Gal; trisaccharides Gal (β 1-6)-Gal (β 1-4)-Glc, Gal-(β 1-3)-Gal (β 1-4)-Glc; tetrasaccharide Gal (β 1-6)-Gal (β 1-6)-Gal (β 1-4)-Glc and pentasaccharide Gal (β 1-6)-Gal (β 1-6)-Gal (β 1-6)-Gal (β 1-4)-Glc.

6. The composition according to claim 1, for use in the prevention or treatment of intestinal inflammatory disorders.

7. The composition according to claim 6, for use in the prevention or treatment of inflammation of the intestinal epithelium.

8. The composition according to claim 6, for use in preventing or reducing TNF-α (Tissue Necrosis Factor) induced inflammation in intestinal epithelium cells.

9. The composition according to claim 6, for use in preventing or reducing TNF-α induced inflammatory response in human enterocytes.

10. A method for the treatment and/or prevention of inflammation comprising orally administering to a mammal an effective amount of an oligosaccharide composition.

11. The method according to claim 10, wherein the composition is a mixture of galactooligosaccharides.

12. The method according to claim 10, wherein the composition is a mixture of galactooligosaccharides comprising disaccharide Gal-Gal, a trisaccharides Gal-Gal-Glc, a tetrasaccharide Gal-Gal-Gal-Glc and a pentasaccharide Gal-Gal-Gal-Gal-Glc.

13. The method according to claim 10, wherein the composition is a mixture of galactooligosaccharides comprising disaccharides Gal (β 1-3)-Glc, Gal (β 1-3)-Gal, Gal (β, 1-6)-Gal, Gal (α 1-6)-Gal; trisaccharides Gal (β 1-6)-Gal (β 1-4)-Glc, Gal-(β 1-3)-Gal (β 1-4)-Glc; tetrasaccharide Gal (β 1-6)-Gal (β 1-6)-Gal (β 1-4)-Glc and pentasaccharide Gal (β 1-6)-Gal (β 1-6)-Gal (β 1-6)-Gal (β 1-4)-Glc.

14. The method according to claim 13, wherein the inflammation is of the intestinal epithelium.

15. The method according to claim 10 for preventing or reducing TNF-α (Tissue Necrosis Factor) induced inflammation in intestinal epithelium cells.

16. The method according to claim 10 for preventing or reducing TNF-α induced inflammatory response in human enterocytes.

17. The method according to claim 10, wherein the mammal is a human.

18. The method according to claim 10, wherein the composition comprises from 1.35 to 9.6 g of the oligosaccharide, preferably from 1.96 to 4.9 g, most preferably 2.7 g.
